(19) United States
(12) Patent Application Publication
(10) Pub. No.: US 2024/0374810 A1
(43) Pub. Date: Nov. 14, 2024

(54) SYSTEM AND METHOD FOR ADMINISTERING ANESTHETIC AGENTS TO A PATIENT

(71) Applicant: QUANTIUM MEDICAL SLU, Mataro (ES)

(72) Inventors: PABLO MARTINEZ VAZQUEZ, Mataro (ES); Erik Weber Jensen, Mataro (ES)

(21) Appl. No.: 18/691,857

(22) PCT Filed: Sep. 14, 2022
(86) PCT No.: PCT/EP2022/075475

§ 371 Date: Mar. 13, 2024

(30) Foreign Application Priority Data
Sep. 14, 2021 (EP) 21382825.4

(51) Int. Cl. A61M 5/142; A61M 5/14; A61M 5/168; A61M 5/172; G16H 20/17

(52) U.S. Cl. CPC A61M 5/142; A61M 5/1407; A61M 5/168; G16H 20/17; A61M 2005/14208; A61M 2005/14296; A61M 2005/1726; A61M 2202/0007; A61M 2202/048; A61M 2205/0294; A61M 2205/10; A61M 2205/3303; A61M 2205/3327; A61M 2205/505; A61M 2230/005; A61M 2230/04; A61M 2230/10; A61M 2230/63; A61M 2230/65

(57) ABSTRACT

A system for administering anesthetic agents to a patient (P) comprises a bio-signal monitor (5) for measuring at least one biological signal on the patient (P). an arrangement of infusion devices (31-33) for infusing at least a first anesthetic agent and a second anesthetic agent to the patient (P). and a control device (2) configured to compute. based on the at least one biological signal. a first index relating to a first effect caused by the first anesthetic agent and a second index relating to a second effect caused by the second anesthetic agent. The control device (2) is configured to compute, based on the first index and the second index. a first setting parameter for adjusting an infusion of the first anesthetic agent and a second setting parameter for adjusting an infusion of the second anesthetic agent.

The invention relates to a system for administering anesthetic agents to a patient according to the preamble of claim 1 and to a method for administering anesthetic agents to a patient.

A system of this kind comprises a bio-signal monitor for measuring at least one biological signal on the patient, for example an electroencephalogram (EEG) signal or an electrocardiogram (ECG) signal or another sensor for measuring any body signal relating to a state of anesthesia. An arrangement of infusion devices, typically placed on a rack at the bedside of a patient, serves for infusing at least a first anesthetic agent and a second anesthetic agent to the patient. A control device is configured to compute, based on the at least one biological signal, a first index relating to a first effect caused by the first anesthetic agent and a second index relating to a second effect caused by the second anesthetic agent.

A system of this kind for example comprises a bio-signal monitor for measuring electroencephalogram (EEG), electromyogram (EMG) and electrocardiogram (ECG) signals, and potentially, in addition, hemodynamic data related to the patient's state of anesthesia such Arterial Pressure (AP), and Cardiac Output (CO). An arrangement of infusion devices, typically placed on a rack at the bedside of a patient, serves for infusing different anesthetic agents under system operation. The practitioner selects which anesthetic agents, depending on their effect, are controlled and/or monitored by the system. From a single controlled agent to two or more different agents each one dedicated to a specific effect on the depth of anesthesia.

Balanced anesthesia is defined as a drug-induced state composed of three major effects on the patient: loss of consciousness (hypnosis), loss of pain sensation or response to noxious stimuli (analgesia) and loss of muscular activity (muscular relation, this latter effect being not always present or induced). In general anesthesia (GA), to obtain the desired effects, anaesthesiologists can use different classes of drugs, mostly hypnotics and analgesics, and neuromuscular relaxants if an effect on muscular activity is needed, allowing patients to undergo surgery and other procedures without the distress and pain they would otherwise experience. The anesthesiologists generally must establish the proper amount of the anesthetic drug agents, for each patient and surgical context, in order to achieve—for each specific effect—a desired level, avoiding under-doses of agents, insufficient drug amount to reach the desired effect level, and over-doses, which are prone to lead to undesired and dangerous side effects.

The state of analgesia for surgery is reached by the administration of analgesics, wherein their demand is estimated for each patient and surgical context. Therefore, there is a need for continuous, preferably non-invasive monitoring of the analgesic effect in the patient. Nociception and the perception of pain define the need for analgesia to obtain pain relief. Automatic responses such as tachycardia, hypertension, emotional sweating and lacrimation, although non-specific, are regarded as signs of nociception and consequently inadequate analgesia.

Similarly to analgesics, when administering a sufficient dose of hypnotics, the resulting loss of consciousness ensures that the patient does not perceive stimuli consciously, but the neuro-vegetative and somatic responses are not necessarily abolished. When administering a sufficient dose of analgesics, nociception stimuli are blocked and neuro-vegetative and somatic responses are prevented. However, analgesics do not necessarily result in a loss of consciousness and amnesia. In summary, analgesia and hypnosis are distinct components or effects of the GA but they are not independent. Rather, there is a link between both, a link that also extends to the third effect, the muscular relaxation.

Anesthesia generally can be regarded as a dynamic process where the balanced effects of the anesthetic drugs are counteracted by the intensity of the different stimuli occurring during surgery. When an equilibrium resulting therefrom is broken, a patient could evolve to a different anesthetic depth, without the anaesthesiologist being aware of it, resulting potentially in an intraoperative awareness of the patient. One of the objectives of modern anesthesia hence is to ensure adequate level of (un-)consciousness to prevent awareness without inadvertently overloading the patient with anesthetics (hypnotics and analgesics), which might cause increased postoperative complications.

There are several widely used clinical methods for assessing the level of consciousness during GA, including the Observer's Assessment of Alertness and Sedation scale (OAAS) and the Ramsey Sedation Scale. However, the disadvantages of using clinical scales in the operating room are that they cannot be used continuously and that they are cumbersome to perform. Furthermore, they require the patient's collaboration, which in some cases might be difficult. This has led to the investigation of an automated assessment of the level of consciousness.

In the last decades, some automatic devices have been introduced into the market to provide an objective quantification of a level of consciousness of the patient, relating to a hypnotic effect. The most prevailing monitoring technologies rely on the electroencephalogram (EEG) where the scalp bioactivity generated in the brain cortex is recorded and subsequently processed to extract distinct EEG features which feed a model or algorithm that maps EEG signal-extracted information into an index, typically in a range of 0 to 100, which it is highly correlated with the hypnotic drug concentration in the brain. EEG features belong either to the frequency domain or the domain of the signal. The models used to map EEG features to the drug concentration range from simple polynomials to more complex functions such as neural networks, with their parameters estimated to give the best fitting.

Recently, a nociception effect related measurement based on the EEG has been proposed by E. W. Jensen et al., “Monitoring hypnotic effect and nociception with two EEG-derived indices, qNOX and qCON, during general anesthesia”, Acta Anaesthesiologica Scandinavica, 2014; 58:933-941. Similarly to the EEG-based hypnosis monitors, this novel index combines a set of different extracted EEG features in a model (implemented either with a quadratic polynomial or with an Adaptive Neuro Fuzzy Inference System (ANFIS) neural network) that maps, under the best fitting, the EEG features summarized as an index, ranging between 0 and 100, with the probability of patient's response to noxious stimuli.

A method for computing an index of nociception is in addition disclosed in WO 2017/012622 A1.

For safe surgical procedures it is crucial for the anaesthesiologist to possess objective methods to estimate continuously the level of each GA component (unconsciousness, analgesia, and immobility) within different phases of the surgical procedure as well as to be able to control the administration of different drugs related with each component. Traditionally, for intravenous anaesthesia, an anesthetic dose-response or dose-effect relationship is described under pharmacokinetics (PK) and pharmacodynamics (PD) models (PKPD). While the PK part describes the drug pharmacokinetic behaviour which is how the drug is distributed through different parts of the body, the drug concentration in the plasma (Cp) representing one the most relevant parameters, the PD part of the model deals with the end response or effect, produced at the biophase or the location where the drug acts. The drug pharmacodynamic behaviour of the drug described by the PD model is decomposed into two parts, one describing the drug concentration at the biophase, named effect-site concentration (Ce), and the other describing the observed effect. For instance, for a hypnotic drug, such as propofol, the PK model part will describe the distribution of the drug in different parts of the body as a function of time, in particular, its plasma concentration, Cp. The PD part models the propofol concentration at the biophase (brain), resulting in an effect site concentration (Ce). Both Cp and Ce are variables in units of concentrations (mass/volume). In addition, the PD part maps the Ce with the observed effect of propofol, for instance, with the consciousness effect evaluated with the (OAAS) and the Ramsey Sedation Scale. The latter is mathematically modelled with a non-linear sigmoidal shape function, Hill model, between the Ce and whatever variable is used to quantify the effect.

While effect measurements, e.g. based on the OAAS or Ramsey scale, are useful for clinical research and punctual estimation of a patient state, these are impractical for continuous state monitoring and drug titration. In that respect, traditionally the Ce concentration functions as a parameter are used to define a specific effect-related target. In this way, the anesthesia practitioner establishes an effect-site concentration (Ce) target for each GA component individually, depending on the surgical context, trying to reach each established target manually or using a target-controlled (TCI) infusion system, which calculates a dose profile scheme for that purpose. However, by defining an effect target by setting the effect-site concentration, from the patient's point of view the system behaves as an open-control system. However, since the introduction of monitoring systems, using indices derived from a patient's bio-signal information and hence providing measures directly related to the patient's state, the practitioners can redefine their desired effect target by setting a proper index target. The monitoring of different effect indices is the key to perform a closed-loop operation of the whole system (including infusion pumps and the patient).

The three basic components of GA (hypnosis or sedation, analgesia or nociception, and immobility or muscular relaxation) are typically not completely independent from each other, but are influencing one another. An overlap between the basic components of GA is generally influenced by physiology as well as the surgical context, by a single or multiple different responses produced by a certain drug, and by the sum of multiple responses from combinations of drugs. For instance, analgesia may be more profound when an analgesic agent is combined with a hypnotic agent, and similarly a deeper hypnosis level may be reached when a hypnotic agent is administered together with an analgesic agent.

Different methods for monitoring nociception are known. For example, a monitoring method by skin conductance has been claimed in U.S. Pat. No. 6,571,124. In another example, U.S. Pat. No. 7,024,234 describes an algorithm that analyses a photoplethysmographic signal for the detection of an autonomic nervous system activity during sleep related breathing disorders. In yet another example, US 2005/143665 A describes a method for assessing a level of nociception during anesthesia by plethysmography from which a number of parameters are derived which are used to design a final index using a multiple logistic regression approach. In yet another example, U.S. Pat. No. 6,685,649 describes a method for detection of nociception by analysis of RR intervals achieved either from ECG data or blood pressure data. From the RR intervals an acceleration emphasized RR interval is defined. In yet another example, US 2009/0076339 A recites a method for monitoring the nociception of a patient during GA by extracting RR intervals from ECG and blood pressure. The method is based on detection of simultaneous increase in HR and BP, defined as a non-baroreflex. In yet another example, EP 1 495 715 A1 recites a method for measuring an index of hypnosis as well as index of analgesia which are independent from each other. Previous nociception methods do not rely on any EEG features.

It is an object of the instant invention to provide a system and method for administering different anesthetic agents, relating to different effects, to a patient within an anesthesia procedure which allows for a reliable, controlled anesthesia procedure while allowing to safely reach desired effects.

This object is achieved by a system comprising the features of claim 1.

Accordingly, the control device is configured to compute, based on the first index and the second index, a first setting parameter for adjusting an infusion of the first anesthetic agent and a second setting parameter for adjusting an infusion of the second anesthetic agent.

The control device is in essence a multi-variate, or multi-input multi-output (MIMO), control system configured to regulate the administration of a set of specific effect-anesthesic agents to a set of desired effect targets. The system can operate from controlling one drug and its desired effect target, to two or more drugs and their corresponding effect targets. For instance, the practitioner can select to control a hypnotic drug, such as propofol, to reach a desired depth of hypnosis (a single drug-single effect control), or add an analgesic agent such as remifentanil (opioid) to control with a desired depth of analgesia (a two drugs/two effects control). Similarly, the system can be extended to operate on more drugs, such as rocuronium (neuro-muscular blocker) with its defined target effect on the muscular relaxation.

In the instant system, the control device is configured to compute from indices which relate to different effects caused by different anesthetic agents and are derived based on one or multiple biological signals, such as EEG, ECG or other signals, the proper controlled pump actions to reach the desired effect based on such indices.

Herein, a control of the infusion takes place based on the different indices. Namely, the control device is configured to compute different setting parameters which serve to control different infusion devices, the computation of the setting parameters being based on the different indices as computed using one or multiple biological signals monitored by the bio-signal monitor. As the indices provide for a quantification of desired effects to be achieved by means of the different anesthetic agents, the control of the infusion devices based on the computed indices allows for an effective control of the effects which shall be achieved during the anesthesia procedure, namely in particular in terms of a hypnotic effect and an analgesic effect.

The first index may in particular be an index of consciousness, for instance an index denoted as qCON. The second index, in turn, may in particular be an index of nociception, for instance an index denoted as qNOX. By means of the indices the hypnotic effect (indicated by the index of consciousness, qCON) and the analgesic effect (indicated by the index of nociception, qNOX) are quantified in a range between 0 and 100. For example in E. W. Jensen et al., “Monitoring hypnotic effect and nociception with two EEG-derived indices, qNOX and qCON, during general anesthesia”, Acta Anaesthesiologica Scandinavica, 2014; 58:933-941 it is described how these indices may be computed.

The index of consciousness, qCON, may for example be computed from the clinical data using a model, for example as described in WO 2017/012622 A1.

The index of nociception, qNOX, may also be computed using a model, in particular a fuzzy logic model (in particular a so-called ANFIS model) or a quadratic model, as for example described in WO 2017/012622 A1. The model, in particular, may be defined by a system of equations using a multiplicity of coefficients for computing said index of nociception from input data derived from an encephalography signal (EEG), wherein the coefficients in a training phase are derived using training data as input to the model. The input to the model are features extracted from the EEG, such as frequency bands, while the output is the corresponding level of nociception assessed by clinical signs. Once the training is completed the model is frozen and may be used, by the control device, during an actual anesthesia procedure to compute the index of nociception during the anesthesia procedure in realtime to provide information with respect to nociception during GA.

The indices, in one embodiment, are repeatedly computed, e.g. continuously with each new measurement of a biological signal of the bio-signal monitor, e.g. with each cardiac cycle. In particular, the different indices may be computed in real-time, such that a real-time feedback based on the indices is obtained.

In one embodiment, the control device is configured to provide the first setting parameter, from a first index value, to a first of the infusion devices for controlling operation of the first of the infusion devices for infusing the first anesthetic agent, and to provide the second setting parameter, from a second index value, to a second of the infusion devices for controlling operation of the second of the infusion devices for infusing the second anesthetic agent. Hence, the control device computes the different indices and, based on the indices, derives the different setting parameters to control the operation of the different infusion devices. The setting parameters are fed to the infusion devices, such that operation of the infusion devices is adapted based on the setting parameters.

Setting parameters are the actions on the infusion rate that the pumps must follow. Hence, by means of the control device, an infusion rate by which a particular anesthetic agent is administered using an associated infusion device is adjusted.

The computation of each setting parameter, indicating the infusion rate to deliver to the patient, herein takes place such that a desired effect, as indicated by a related index, is achieved. Hence, within the system a control loop may be formed, the control device computing setting parameters to adapt the operation of the infusion devices in a way that the indices are controlled to converge to predefined target values or ranges.

The system, in one embodiment, comprises a switch module that is actuatable to switch between two configurations, namely the closed-loop configuration and an advisory (or open-loop) configuration. Herein, in the closed-loop configuration, the control device is configured to automatically output the first setting parameter to the first of the infusion devices and the second setting parameter to the second of the infusion devices. In contrast, in the advisory configuration, the control device is configured to output the first setting parameter and the second setting parameter to an user for a confirmatory input, using a touch display, prior to transferring the first setting parameter to the first of the infusion devices and the second setting parameter to the second of the infusion devices.

n the closed-loop configuration an automatic, closed control loop is established, the control device feeding the computed setting parameters to the infusion devices such that the infusion devices are controlled to achieve effects in the patient according to predefined targets of the different indices. In the open-loop configuration, the control device just as well computes the setting parameters, but displays the setting parameters, for example to the practitioner, such that the practitioner may confirm the setting parameters. In the open-loop configuration, hence, the system acts as an advisory system in which the control device computes setting parameters for controlling the operation of the infusion devices, wherein the setting parameters are fed to the infusion devices only upon explicit confirmatory input by a user, wherein the user, under its knowledge and responsibility, may also follow or modify the setting parameters.

In one embodiment, PKPD dose-effect models for each effect and drug under consideration are used. Based on these models corresponding indexes per effect are derived from bio-signals.

In one embodiment, the control device is configured to compute the first setting parameter and the second setting parameter based on a first PKPD model relating to a PKPD behaviour of the first anesthetic agent and a second PKPD model relating to a PKPD behaviour of the second anesthetic agent. By means of PKPD model an effect-site concentration may be estimated in dependence of an input dose. Inversely, from a (known) effect-site concentration it may be computed in which way the input dose should be adjusted to obtain a desired effect-site concentration. The desired effect-site concentration herein may be computed based on the indices.

Generally, each anesthetic agent exhibits different PKPD behaviours, which are modelled with differential linear equations representing the drug concentration in different virtual body compartments. The PKPD model parameters are functions of patient characteristics, such as age, gender, height and weight, and are provided in the literature for each drug. The model herein may e.g. use three compartments, which is the most common model of anesthetic agents in GA. The PK part of the model describes the drug diffusion in several compartments, representing different parts of the body, from a central compartment or volume describing the drug diffusion in the plasma/blood volume, to other compartments modelling muscle and fat masses as well as the most relevant organs involved in the drug metabolism such as kidney and liver. The dynamical evolution of the concentration in each compartment with the interactions between compartments is described using differential equations which model the processes of resorption, diffusion, biochemical metabolism and excretion. The more compartments are added, the better the specific modelling per organ or function, but in general simple PK models relying on 3 or 4 compartments may provide acceptable results for the most relevant parameter evaluated in the PK part, the plasma concentration (Cp). While the drug distributes through the blood/plasma and the other compartments, the drug takes some time to reach the brain, which is the biophase or action area of the drug. The drug diffusion from the plasma to the brain, quantified with the effect site concentration (Ce), is modelled by the first part of the PD model with a linear differential equation that considers the brain as another extra compartment connected to the central volume. Altogether, for a given anesthetic agent and infusion rate (r(t), as a function of time), the corresponding linear differential equation system models the concentrations of the anesthetic agent in each compartment, in particular, the plasma concentration (Cp) and an effect-site concentration (Ce).

A set of linear differential equations of the PKPD model for a certain anesthetic agent may summarized as:

$$\dot{x} ( t ) = A x ( t ) + B r ( t )$$

The PKPD model takes into account all diffusion mechanisms of the drug across the compartments or equivalent body parts, inter-compartmental clearance, and diverse mechanisms of the drug elimination. A state-vector

$$x ( t ) = [ \begin{matrix}{C}_{1} & {C}_{2} & {C}_{3} & Ce\end{matrix} ] ( t )$$

describes the drug evolution in the different body compartments and a virtual compartment for modelling the bio-phase concentration Ce, where the first compartment concentration refers to the drug concentration in plasma, therefore C₁(t) is equal to Cₚ(t). The other concentrations C₂ and C₃ apply to the other compartments, in this case, 2 and 3 describing some areas and organs.

Matrices A and B include PKPD model parameters, which are constants proportional to diffusion rates between compartments as well as metabolism and excretion. The constants are dependent on patient characteristics, such as age, gender, height and weight. Definitions for the constants are reported in the literature for each drug. One advantage of modelling the anesthetic agent in the body with a system of differential linear equations is that there is an analytical solution to the set of equations, according to:

$$x ( t ) = C Φ ( t ) + Φ ( t ) {\int }_{t 0}^{t} {Φ}^{- 1} ( s ) B r ( s ) dsdt \vert$$

$$where$$

$$Φ ( t ) = {e}^{At}$$

is an exponential matrix term.

The sigmoidal-wise Hill equation, θ=θ(Ce), allows to compute an effect from a drug concentration at an effect-site. Hence, using an inverse Hill equation the effect-site concentration can be derived from the effect, which in the instant case is expressed by the respective index value. The Hill equation generally can be formulated as follows:

$$Effect = θ ( C e ) = {E}_{0} - \frac{{E}_{max} {C}_{e}^{γ}}{{C}_{50}^{γ} + {C}_{e}^{γ}}$$

where Cₑ is the drug effect-site concentration, and E₀ and Eₘₐₓ are the minimum effect (no drug, Cₑ=0) and the maximum effect, respectively. The slope and sigmoidicity of the Hill function is given by the variable γ (>0), and the inflection point location correspond to the C₅₀ value, which corresponds with the steady drug concentration producing half the maximum effect.

In one embodiment, the control device is configured to compute the first setting parameter and the second setting parameter based on a first Hill model, modelling a first effect at an effect-site based on an effect-site concentration of the first anesthetic agent and a second Hill model modelling a second effect at the effect-site based on an effect-site concentration of the second anesthetic agent. The first effect, caused by the first anesthetic agent, in particular, is indicated by the first index, whereas the second effect, caused by the second anesthetic agent, is indicated by the second index. Having computed (momentary) values for the first index and the second index, hence, conclusions with respect to the effect-site concentration can be drawn, wherein for example values for the effect-site concentration of the first anesthetic agent and the second anesthetic agent may be computed using inverse Hill equations with the first index and the second index as input. Hence, using a (first) inverse Hill equation with the first index as input, an effect-site concentration of the first anesthetic agent can be estimated. Likewise, using a (second) inverse Hill equation with the second index as input, an effect-site concentration of the second anesthetic agent can be estimated. Hence, from the momentary values of the first index and the second index resulting effect-site concentrations of the first anesthetic agent and the second anesthetic agents may be modelled. Based on the effect-site concentrations, then, using respective pharmacokinetic/pharmacodynamic models the setting parameters can be determined.

The Hill equation herein is adapted for the specific anesthetic agent, such that for each anesthetic agent a corresponding Hill equation is defined, for example in an initial training phase.

In one embodiment, the control device is configured to compute the first setting parameter and the second setting parameter based on an interaction model modeling a combined effect of an effect-site concentration of the first anesthetic agent and an effect-site concentration of the second anesthetic agent. Hence, when computing the setting parameters also an interaction effect of the anesthetic agents is considered. This is based on the finding that different anesthetic agents interact in that an effect caused by one anesthetic agent is influenced by another anesthetic agent. For example, an analgesic effect may be more profound when an analgesic agent is combined with a hypnotic agent, and a deeper hypnosis level may be reached when a hypnotic agent is administered together with an analgesic agent.

Generally, a common effect due to an interaction of the different drugs can be modelled as a 2D sigmoid-wise function of two effect-site concentrations formulated using Minto's interaction model as follows:

$$Effect = H ( C {e}_{A} , {Ce}_{B} ) = \frac{{( \frac{{C}_{A} + {C}_{B}}{{C}_{50} ( T )} )}^{γ}}{1 + {( \frac{{C}_{A} + {C}_{B}}{{C}_{50} ( T )} )}^{γ}}$$

$$where$$

$$T = ( {C}_{A} ) / ( {C}_{A} + {C}_{B} )$$

$${C}_{50} ( T ) = 1 - β T + β {T}^{2}$$

In the above equations, C_{A}, C_{B} are the normalized effect-site concentrations of the anesthetic agents A and B to their respective potency concentration, γ is the steepness of the relation between the drug combination and the effect measured as probability, and β describes the interaction strength. The parameters γ, β are obtained by fitting the input-output variables over a large dataset of patients during an initial training phase to train the model.

Similarly, the interaction between two drugs can be described with the same parametric surface formulation but the input variables consisting of the indexes quantifying the effects according to

Effect=I(index A, index B)

where equivalently to the H function

$$S = ( {index}_{A} ) / ( {index}_{A} + {index}_{B} )$$

$${I}_{50} ( S ) = 1 - β S + β {T}^{2}$$

Herein, I₅₀ corresponds to the steady drug concentration producing half the maximum effecet.

Accordingly, γ, β are obtained from fitting the model to an extensive dataset of two indexes during GA. Both formulations are correlated, and their main difference relies on their input space domain, the effect-site space for the H function and the index space for the I function.

Generally, the control is based on two types of parametric models: an individual sigmoidal-wise drug dose-response parameterization, such as Hill model, binding each drug effect-site concentration, given by its pharmacokinetic/pharmacodynamic model, to its corresponding bio-signal based index, and a multivariate sigmoidal-wise interaction model, such as the Minto's interaction model.

The control device, in one embodiment, is configured—under the administration of two anesthetic agents—to provide the proper multivariate controls, where the pumps setting parameters are based on the two drugs parametric interaction model and the individual dose-response/effect parametric models with their corresponding bio-signal based indexes values.

In one embodiment, the control device is configured—under the administration of a single anesthetic agent—to provide a single pump setting parameter based on the drug's dose-response/effect parametric model with its corresponding bio-signal based index value.

In one embodiment, the control device may be switchable between a multi-drug operation, in which multiple anesthetic agents are administered to a patient, and a single-drug operation, in which a single anesthetic agent is administered to a patient.

In one embodiment, the control device is configured to operate with the parametric single dose-response/effect and the interaction model in combination with a multivariate controller, such as a PID or LQR, for defining the pump setting parameters.

In one embodiment, the bio-signal monitor comprises an EEG monitor for measuring an electroencephalogram signal on the patient.

Alternatively or in addition, the bio-signal monitor comprises an EMG monitor for measuring an electromyography signal on the patient.

Yet alternatively or in addition, the bio-signal monitor comprises an ECG monitor for measuring an electrocardiogram signal.

Yet alternatively or in addition, the bio-signal monitor comprises a hemodynamics monitor for measuring at least one signal relating to hemodynamics of the patient, for example, blood pressure, cardiac output or the like.

Yet alternatively or in addition, the bio-signal monitor comprises an impedance monitor for measuring a bio-impedance on the patient.

Yet alternatively or in addition, the bio-signal monitor comprises a plethysmography sensor, a piezoelectric sensor, and/or a skin response sensor.

By means of the bio-signal monitor, biological signals are measured, and from the biological signals the different indices are computed. Herein, an index may be computed based on a single biological signal, such as an EEG or ECG signal. Alternatively, a combination of multiple biological signals, such as an EEG signal and an ECG signal, a hemodynamic signal, an impedance signal and a signal of a plethysmography sensor, may be taken into account for computing one or multiple indices.

Generally, e.g. nociception and the perception of pain define the need for analgesia for obtaining pain relief. The state of analgesia for surgery is reached by the administration of analgesics. The demand of analgesics is individual for each patient, such that there is a need for continuous, preferably non-invasive monitoring of the analgesia of the patient. Autonomic responses such as tachycardia, hypertension, emotional sweating and lacrimation, although non-specific, are regarded as signs of nociception and consequently inadequate analgesia. Hence, by means of the bio-signal monitor signals relating to different bio-states may be measured, for example relating to a tachycardia, hypertension, emotional sweating and lacrimation, such that such measurements may be taken into account for computing a suitable index.

In one embodiment, the bio-signal monitor comprises a stimulation device for applying at least one stimulus to the patient. By means of the stimulation device an (external) stimulus may be caused on the patient, and a response of the patient may be monitored.

The stimulation device in particular may be configured to apply different stimuli to different locations of the patient. In particular, stimuli may be applied to different nerve regions of the patient, such as around the palpebral nerve and around the radian/median nerve. The stimuli herein may differ depending on the different locations, the stimuli for example exhibiting different frequencies and durations, wherein the stimuli may be controlled independently.

In one embodiment, the stimulation device is configured to apply an electrical stimulus, for example by injecting a stimulation current into the patient using one or multiple electrodes placed on the patient. Stimulation currents may have an intensity in a range between 1 to 50 mA, assuming a 1 kΩ load, wherein a stimulus may be applied according to a predefined pulse pattern with pulse durations between 10 to 1000 μs and with a frequency in between 1 to 250 Hz.

Using the stimulation device stimuli may be caused on the patient, and a response may be monitored, wherein the response may be taken into account for computing at least one of the indices.

By means of the system, more than two anesthetic agents may be administered to the patient. For example, a third infusion device may be used to deliver a third anesthetic agent to the patient, for example a muscular relaxant to cause immobility by muscular relaxation of the patient. The control device herein may be configured to compute, based on the at least one biological signal, relying mainly on the EMG activity, a third index relating to a third effect caused by the third anesthetic agent infused by the arrangement of infusion devices, wherein the control device may be further configured to compute, based on the third index, a third setting parameter for adjusting an infusion of the third anesthetic agent.

Similar as for the first and the second anesthetic agent, the system may be operated in an advisory (or open-loop) configuration or a closed-loop configuration, the control device being configured to feed the third setting parameter to the third infusion device automatically (in a closed-loop configuration) or only upon explicit user input (in an open-loop configuration in which the system acts as an advisory system).

It shall be noted that the system is not limited to the use of three anesthetic agents. Rather, also more than three anesthetic agents may be delivered, wherein the administration of the anesthetic agents may also be controlled by means of the control device by computing suitable indices and by controlling the respective infusion devices making use of the indices and by controlling the respective infusion devices making use of the indices.

The object is also achieved by means of a method for administering anesthetic agents to a patient, the method comprising: measuring at least one biological signal on the patient using a bio-signal monitor; infusing at least a first anesthetic agent and a second anesthetic agent to the patient using an arrangement of infusion devices; computing, using a control device and based on the at least one biological signal, a first index relating to a first effect caused by the first anesthetic agent and a second index relating to a second effect caused by the second anesthetic agent; and computing, using the control device and based on the first index and the second index, a first setting parameter for adjusting an infusion of the first anesthetic agent and a second setting parameter for adjusting an infusion of the second anesthetic agent.

The advantages and advantageous embodiments described above for the system equally apply also to the method, such that in that respect it shall be referred to the above.

The idea underlying the invention shall subsequently be described in more detail by referring to the embodiments shown in the figures. Herein:

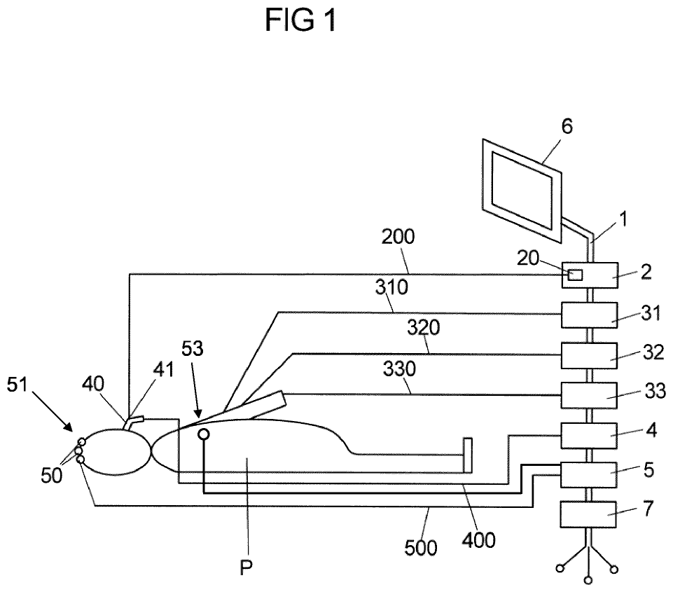

FIG. 1 shows a schematic view of a setup in an anesthesia procedure;

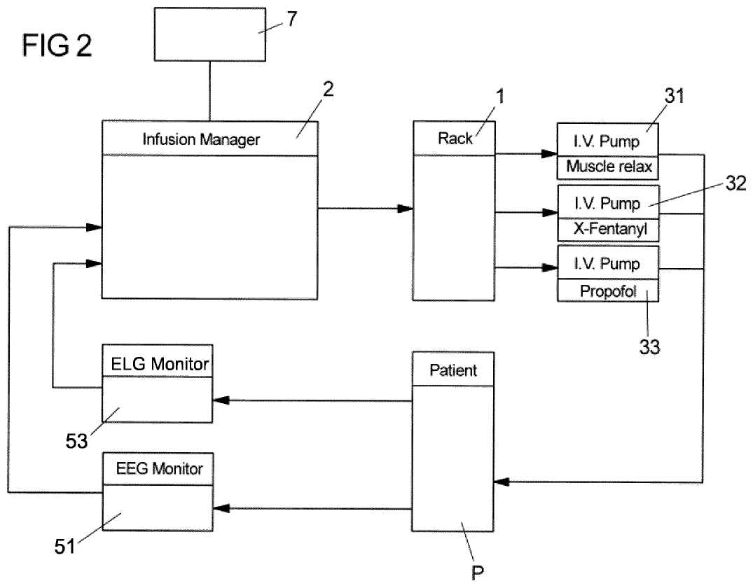

FIG. 2 shows a functional diagram of the setup of FIG. 1;

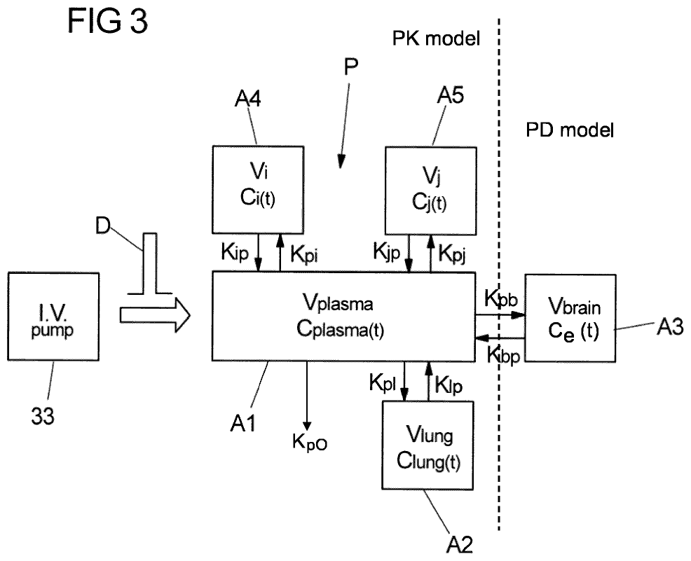

FIG. 3 shows a functional diagram of a model for modelling the distribution of a drug dosage in a patient's body;

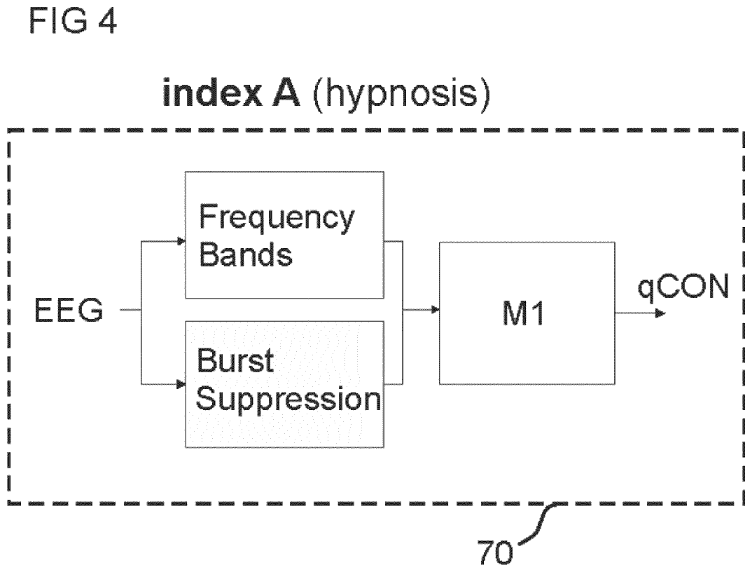

FIG. 4 shows a schematic drawing of a model for computing an index of consciousness (qCON);

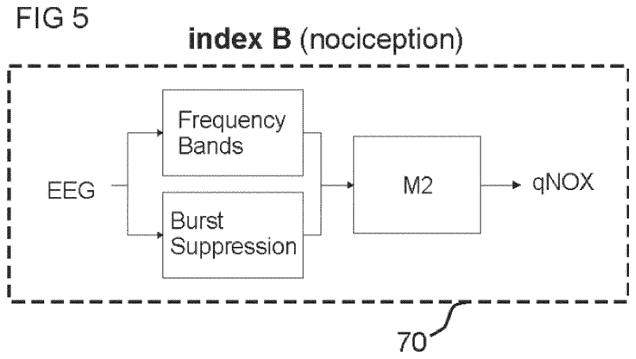

FIG. 5 shows a schematic drawing of a model for computing an index of nociception (qNOX);

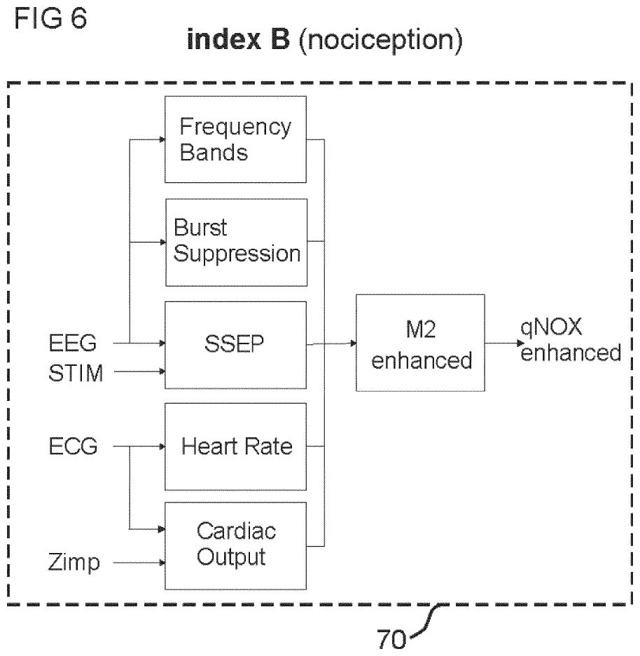

FIG. 6 shows a schematic drawing for a correction of a value for the index of nociception as output by the model;

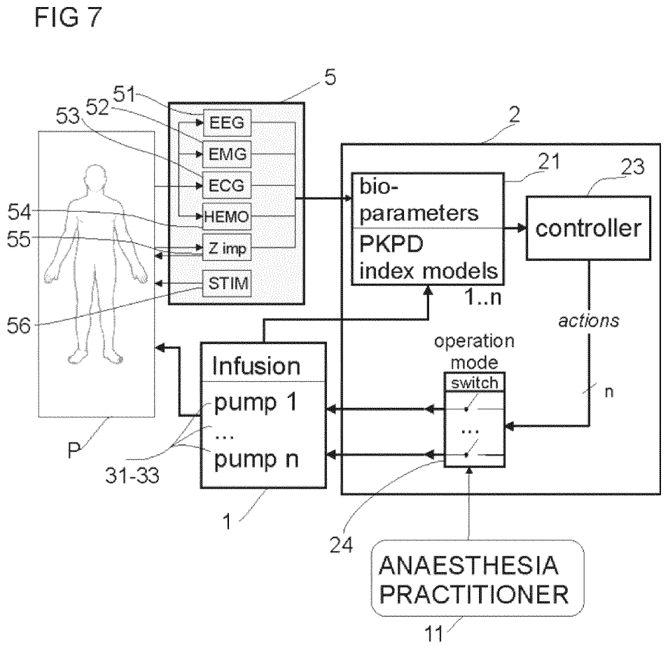

FIG. 7 shows a schematic drawing of an embodiment of a system for controlling an anesthesia procedure involving multiple anesthetic agents;

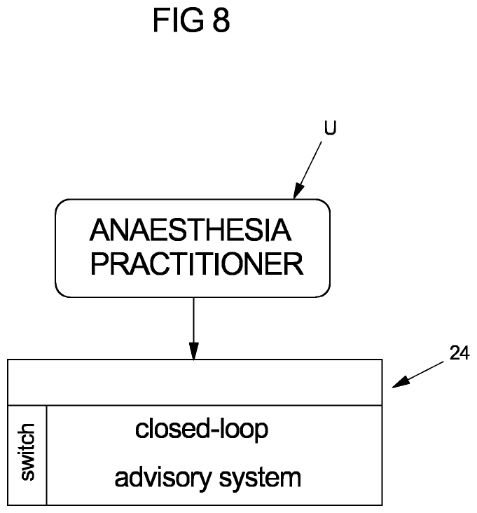

FIG. 8 shows a schematic drawing of a switch module for switching between a closed-loop configuration and an open-loop configuration;

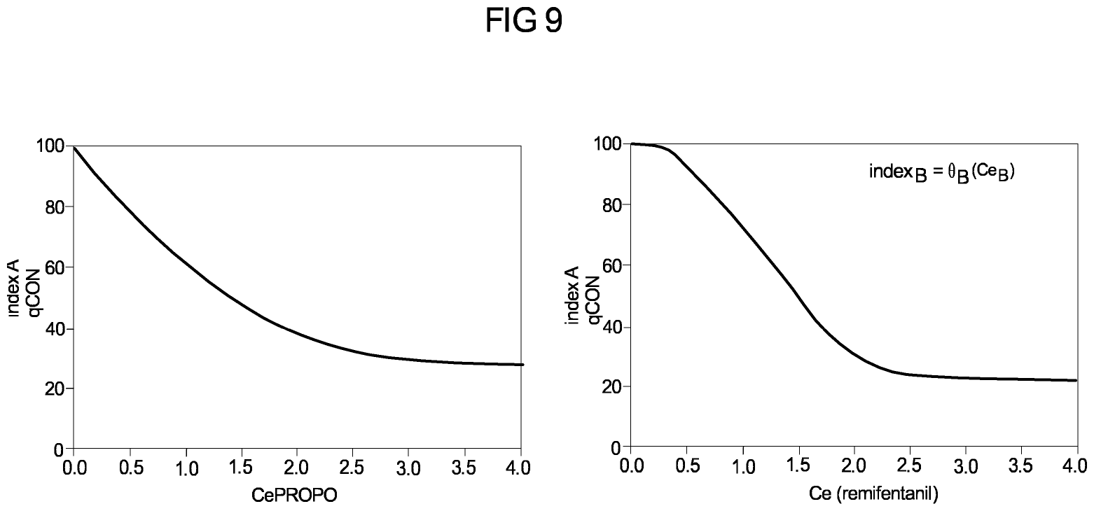

FIG. 9 shows a view of a computed index as a function of an effect-site concentration;

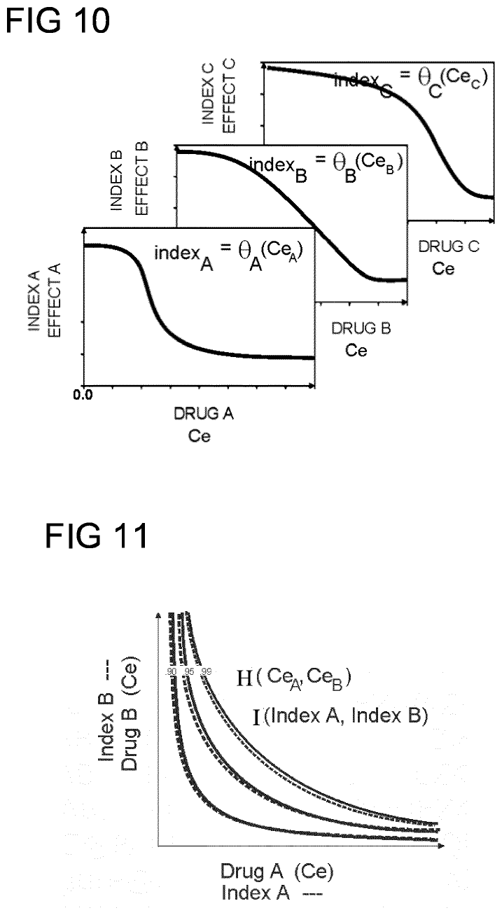

FIG. 10 shows different indices as a function of the effect-site concentrations of the different anesthetic agents independently associated with the different indices;

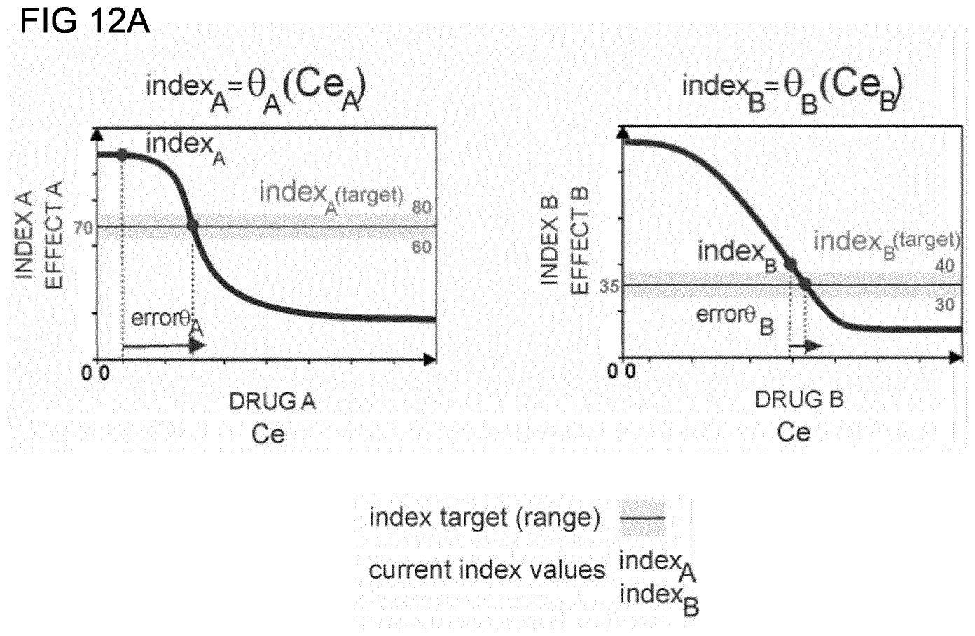

FIG. 11 shows the interaction functions H and I, with the effect-site concentrations or the index values as inputs, respectively;

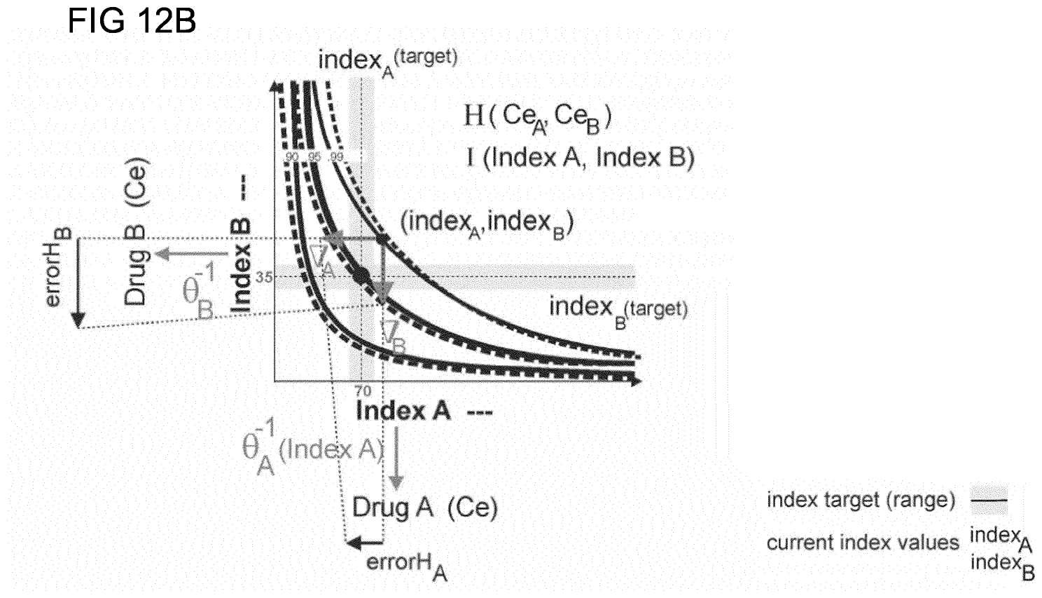

FIG. 12 illustrates how error signals between a current state and the targets are derived;

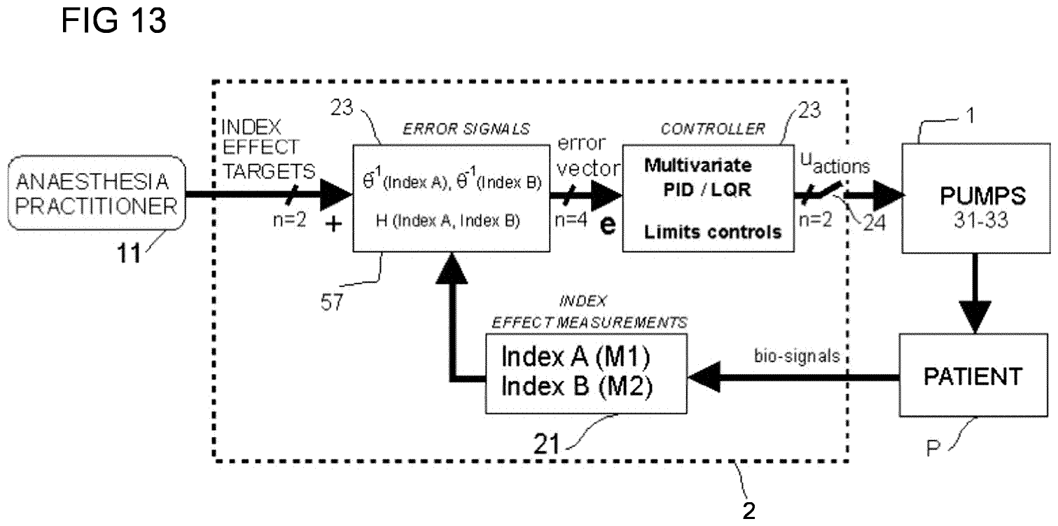

FIG. 13 shows a schematic view of an embodiment of a controller module for controlling the operation of infusion devices for administering the different anesthetic agents; and

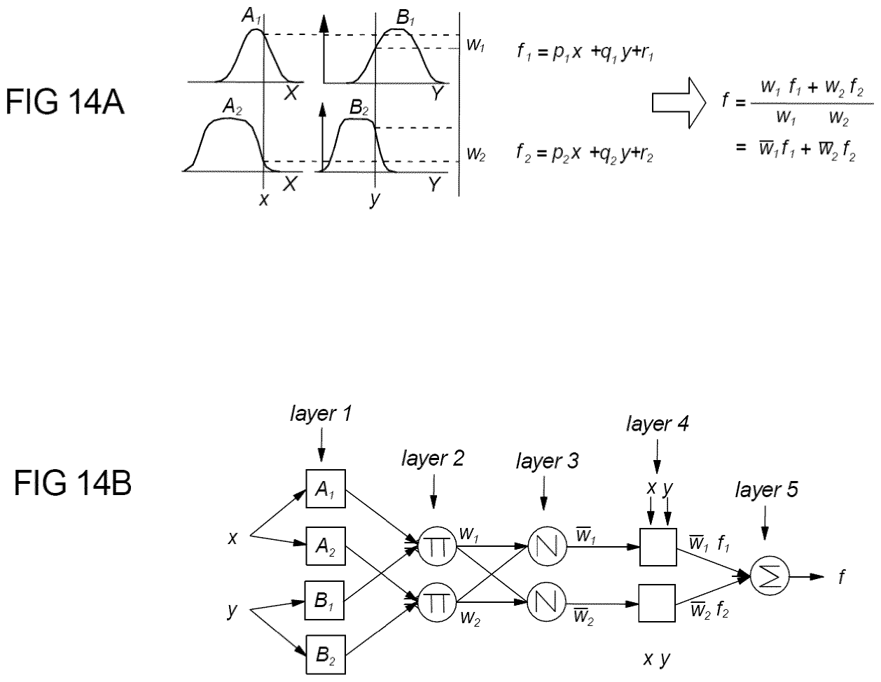

FIG. 14A, 14B show a mathematical formulation of an ANFIS non-linear model.

Subsequently, a system and method for administering anesthetic agents to a patient in an anesthesia procedure in a controlled fashion, allowing for an automatic operation, shall be described in certain embodiments. The embodiments described herein shall not be construed as limiting for the scope of the invention.

Like reference numerals are used throughout the figures as appropriate.

FIG. 1 shows a schematic drawing of a setup as it generally is used for example in an anesthesia procedure for administering an anesthetic drug such as propofol and/or remifentanil to a patient P. In this setup multiple devices are arranged on a rack 1 and are connected via different lines to the patient P.

In particular, infusion devices 31, 32, 33 such as infusion pumps, in particular syringe pumps and/or volumetric pumps, are connected to the patient P and serve to intravenously inject, via lines 310, 320, 330, different drugs such as propofol, remifentanil and/or a muscle relaxant drug to the patient P in order to achieve a desired anesthetic effect. The lines 310, 320, 330 are for example connected to a single port providing access to the venous system of the patient P such that via the lines 310, 320, 330 the respective drugs can be injected into the patient's venous system.

The rack 1 furthermore may hold a ventilation device 4 for providing an artificial respiration to the patient P while the patient P is under anesthesia. The ventilation device 4 is connected via a line 400 to a mouth piece 40 such that it is in connection with the respiratory system of the patient P.

The rack 1 also holds a bio-signal monitor 5 including, for example, an EEG monitor 51 and an ECG monitor 53, the bio-signal monitor e.g. being adapted to sense signals by means of electrodes attached to the patient's body for monitoring signals on the patient during an anesthesia procedure.

In addition, a control device 2 is held by the rack 1. The control device 2 serves to control the infusion operation of one or multiple of the infusion devices 31, 32, 33 during the anesthesia procedure such that infusion devices 31, 32, 33 inject anesthetic drugs to the patient P in a controlled fashion to obtain a desired anesthetic effect. This shall be explained in more detail below.

FIG. 2 shows a functional diagram of a control loop for controlling the infusion operation of the infusion devices 31, 32, 33 during an anesthesia procedure. The control loop herein may in principle be set up as a closed-loop in which the operation of the infusion devices 31, 32, 33 is automatically controlled without user interaction. Alternatively, the system is set up as an open-loop system in which at certain points of time, in particular prior to administering a drug dosage to a patient, a user interaction is required in order to manually confirm the operation.

The control device 2, also denoted as “infusion manager”, is connected to the rack 1 which serves as a communication link to the infusion devices 31, 32, 33 also attached to the rack 1. The control device 2 outputs control signals to control the operation of the infusion devices 31, 32, 33, which according to the received control signals inject defined dosages of drugs to the patient P.

By means of the bio-signal monitor 5 e.g. in the shape of an EEG monitor for example an EEG reading of the patient P is taken. The measured data obtained by the bio-signal monitor 5 are fed back to the control device 2, which correspondingly adjusts its control operation and outputs modified control signals to the infusion devices 31, 32, 33 to achieve a desired anesthetic effect.

The control device 2 uses, to control the infusion operation of one or multiple infusion devices 31, 32, 33, a pharmacokinetic-pharmacodynamic (PK/PD) model, which is a pharmacological model for modelling processes acting on a drug in the patient's P body. Such processes include the infusion, the distribution, the biochemical metabolism, and the excretion of the drug in the patient's P body (denoted as pharmacokinetics) as well as the effects of a drug in an organism (denoted as pharmacodynamics). Preferably, a physiological PK/PD model with N compartments is used for which the transfer rate coefficients have been experimentally measured beforehand (for example in a proband study) and are hence known. To simplify the PK/PD model not more than 4-5 compartments preferably are used.

A schematic functional drawing of the setup of such a PK/PD model p is shown in FIG. 3. The PK/PD model p logically divides the patient P into different compartments A1-A5, for example, a plasma compartment A1 corresponding to the patient's P bloodstream, a lung compartment A2 corresponding to the patient's P lung, a brain compartment A3 corresponding to the patient's P brain and other compartments A4, A5 corresponding, for example, to muscular tissue or fat and connective tissue. The PK/PD model p takes into account the volume V_{Lung}, Vₚₗₐₛₘₐ, V_{brain}, Vᵢ, Vⱼ of the different compartments A1-A5 as well as transfer rate constants K_{PL}, K_{LP}, K_{BP}, K_{PB}, K_{IP}; K_{PI}, K_{JP}, K_{PJ} indicating the transfer rates between the plasma compartment A1 and the other compartments A2-A5, assuming that a drug dosage D by means on an infusion device 33 is injected into the plasma compartment A1 and the plasma compartment A1 links the other compartments A2-A5 such that an exchange between the other compartments A2-A5 always takes place via the plasma compartment A1. Kp0 describes a constant rate for processes acting through metabolism or elimination that irreversible remove the drug from the central compartment. The PK/PD model p serves to predict the concentration C_{lung}, Cₚₗₐₛₘₐ, Cₑ, Cᵢ, Cⱼ of the injected drug in the different compartments A1-A5 as a function of time.

During a GA procedure, carried out for example by using a control device 2 and a control in the sense of a target-controlled infusion (TCI), it generally is desired to be able to provide for an accurate assessment of an anesthetic state of a patient. For this, from information obtained during a GA procedure, e.g. from EEG signals obtained from the bio-signal monitor 5, indices shall be computed reflecting e.g. a level of consciousness and a level of nociception of a patient during the anesthesia procedure.

This is schematically illustrated in FIGS. 4 and 5. Namely, based on an EEG signal obtained during a GA procedure a first model M1 may be used to compute an index of consciousness qCON (FIG. 4), and a second model M2 may be used to compute an index of nociception qNOX (FIG. 5), as this is described for example in WO 2017/012622 A1. During a GA procedure, hence, the models M1, M2 are used to compute from an input EEG signal an index of consciousness qCON reflecting a level of consciousness, and an index of nociception qNOX reflecting a probability of response to noxious stimuli. Both indices generally have values ranging from 0 to 100 (where higher values indicate an increased consciousness and nociception, respectively).

For computing the index of nociception qNOX, EEG data is fed to the model M2, and a value for the index of nociception qNOX is obtained as output from the model M2.

Herein, in addition to the qNOX definition described WO 2017/012622 A1, an enhancement of the qNOX index, qNOXenhanced, may be employed by extending its formulation based on the EEG with several additional biosignals related to the nociception. FIG. 6 shows the scheme for the qNOXenhanced index, where a model M2enhanced is defined as a function of previous used EEG information, in M2 model for qNOX, and several nociceptive complementary parameters extracted such as the heart rate obtained from the ECG activity, the cardiac output obtained from both, ECG and impedance cardiography Zimp, and somatosensory evoked potential (SSEP) parameters embedded in the EEG activity that are time-locked to an external electrical stimulus.

The model M2 to compute the index of nociception may for example be a quadratic model or a fuzzy logic model, in particular an ANFIS model as shall be subsequently be described in more detail according to different examples. Generally, the model M2 may be represented by a system of equations comprising a multiplicity of coefficients, which are suitably defined in an initial training phase by training the model M2 such that the model M2 reliably provides an output for the index of nociception when feed with input data derived from the EEG signal, as illustrated in FIG. 5.

Based on computed indices, it herein is proposed to control operation of the infusion devices 31-33 to administer different anesthetic agents to the patient P. In particular, based on biological signals as measured by the bio-signal monitor 5 index values relating to different effects caused by the different anesthetic agents may be computed, and based on the indices setting parameters may be determined for controlling operation of the infusion devices 31-33 to adjust the infusion operation for causing the different indices to converge towards predefined targets. As the control takes place based on indices relating to different effects, anesthesia is controlled with respect to specific effects, such that a reliable and safe procedure can be established.

Referring now to FIG. 7, in a general setup, the bio-signal monitor 5 may comprise an EEG monitor 51 for measuring an electroencephalogram signal, an ECG monitor 53 for measuring an electrocardiogram signal, an EMG monitor 52 for measuring an electromyography signal, a hemodynamics monitor 54 for measuring hemodynamic signals, such as the blood pressure or cardiac output, an impedance monitor 55 for measuring bio-impedance signals, and a stimulation device 56 for causing a stimulus on the patient P to measure a corresponding response. Further sensors may be present, such as motion sensors, for example, a piezoelectric sensor, for detecting motion for example in response to a stimulus applied to the patient P, a skin response sensor, a plethysmography sensor or the like.

In the embodiment shown in FIG. 7, the control device 2 comprises an extraction module 21 which serves to extract and compute indices based on bio-signals obtained from the bio-signal monitor 5.

Generally, two or more indices can be computed which relate to different effects caused by different anesthetic agents. In a setup in which for example two infusion devices 31-33 are used to administer two different anesthetic agents to the patient P (for example a hypnotic agent and an analgesic agent), two indices—which generally are referred to as index A and index B—may be computed, the first index A, for example, indicating an effect caused by the first anesthetic agent, for example, the hypnotic agent, and the second index B indicating an effect caused by the second anesthetic agent, for example, the analgesic agent.

The indices may be computed based on bio-signal measurements of the bio-signal monitor 5, for example, based on an EEG signal (with or without applying a stimulus by means of the stimulation device 56), an ECG signal, hemodynamics parameters, an impedance signal, and for example a plethysmography signal.

For example, index A relating to the effect of the first anesthetic agent, for example, a hypnotic agent, in the shown embodiment is computed entirely from the spontaneous EEG signal. As index A, reflecting the hypnosis depth, the qCON index is used, as described in WO 2017/012622 A1. In summary, the qCON index is defined as the output of a quadratic or ANFIS model whose parameters give the best fitting between several inputs, consisting in a set of EEG frequency bands and the percentage of quasi-isoelectric EEG periods for a given time, named Burst Suppression, to an output scalar that best correlates with the hypnotic drug (e.g. propofol) concentration Ce at the effect site. Posteriorly, the model output scalar is normalized to the range of 0 to 100 according to the index definition. The best fitting estimations of the model parameters are obtained from a dataset of EEG data and hypnotic drugs PKPD information collected from a large number of patients during GA during an initial training phase for training the model.

This may be summarized by the following equation:

$$index A = M 1 ( EEG ) = qCON ( EEG bands , EEG Burst Suppression )$$

Index B, in one embodiment, is defined as the index qNOX, as described in WO 2017/012622, and is obtained as an output of a quadratic or ANFIS model whose parameters give the best fitting between several inputs, consisting in a set of EEG frequency bands and the percentage of quasi isoelectric EEG periods for a given time, named Burst Suppression, to an output scalar that best correlates with the analgesic drug effect-concentration, e.g. remifentanil Ce. Posteriorly, the model output scalar is normalized to the range of 0 to 100, according to the index definition. In this case, index B is obtained (only) from the EEG information. In such definition, the index B can be summarized mathematically as

$$index B = M 2 ( E E G ) = q NOX ( EEG bands , EEG Burst Suppression )$$

In another embodiment, index B reflecting the level of analgesia is reformulated by extending the qNOX definition with extra parameters extracted from more biosignals and electrical stimulation, as this is illustrated in FIG. 6. The enhanced qNOX is a function of the EEG and (1) the evoked EEG activity (Somato-sensory evoked potential, SSEP) obtained by averaging the EEG activity at the time electrical stimuli are given, (2) the heart rate extracted from the ECG signal, (3) and the cardiac output obtained from the ECG and the impedance signal. The index B formulated in terms of its bio-signals is:

index B=M2(EEG, SSEP, ECG, Zimp)

The electrical stimulation to elicit the SSEP denotes a response to controlled electrical stimuli between 1 to 50 mA (at 1 kΩ load) using a predefined pulse pattern with pulse durations between 10 to 1000 μs and with a frequency in between 1 to 250 Hz, stimulating either the palpebral nerve or a hand nerve such as the radial nerve.

The features extracted from the EEG and used to feed the model are, in one embodiment, the same as the ones used according to the previous definition of qNOX, namely a set of frequency bands and a burst suppression measurement.

The features extracted from the SSEP, which is obtained by a stimulus time-locked EEG average, are the amplitudes and latencies of the SSVEP components (N25, P60, N80), as well as a measure defined as the norm of the SSVEP derivate

$$iSSVEP = {( {\sum }_{i = 1}^{N} {( S S V E P [ i ] - SSVEP [ i + 1 ] )}^{2} )}^{\frac{1}{2}}$$

The heart rate and the cardiac output are obtained by standard procedures using the ECG and the impedance amplitude and norm of the derivative.

Summarizing, the enhanced definition of index B, assigned to the analgesia effect, is as follows:

index B=M2(EEG frequency bands, SSVEP peak amplitudes, SSVEP peak latencies, heart rate, cardiac output, Zimp amplitude, Zimp norm derivative)

If another, third anesthetic agent, for example, a muscular relaxant, is administered to the patient, a third index, index C, may be added to the multivariate controller to be controlled with the previous indexes A and B. This index relating to the (im-)mobility of the patient may be derived from a third model M3 whose inputs are the facial EMG obtained from energy of the EEG signal above 40 Hz and the norm of the averaged instant velocity and acceleration given by an accelerometer, placed on a muscle innervated by the electrical stimuli used to elicit the SSVEP used in the enhanced index B definition. Thus, the definition of index C requires an electrical stimulation.

The index C in terms of its input signals can be formulated as:

Index C=M3(EEG, accelerometers)

or specifically as

$$index C = M 3 ( energy EEG band > 40 Hz , norm ( v ) , norm ( a ) )$$

The model parameters are adjusted to an index in a range between 0 to 100, giving the best fit to the probability of movement observed in a large dataset of patients under GA with muscular relaxants. The energy EEG band >40 Hz modulates accelerometer responses not related with the electrical stimulus-response.

The indices as derived by the extraction module 21 are fed to a controller module 23, together with an output from a modelling module 22 in which the pharmacokinetic/pharmacodynamic behaviour of the different anesthetic agents is modelled within the different compartments of the patient P. The controller module 23 then computes setting parameters, which are fed towards the infusion devices 31-33 to adapt the operation of the infusion devices 31-33, in particular a dose rate by which the different anesthetic agents are administered to the patient P.

The system as depicted in FIG. 7 herein may be operated in a closed-loop configuration or in advisory configuration. For this, a switch module 24 is provided, by means of which a user, in particular an anesthesia practitioner U, may choose between the different configurations. In the closed-loop configuration, the setting parameters as computed by the controller module 23 are directly fed to the infusion devices 31-33. In the open-loop configuration, in turn, the system functions as an advisory system in which the setting parameters are determined by the controller module 23 and are displayed to the user U for confirmation, wherein the setting parameters are fed to the infusion devices 31-33 only upon confirmation by the user U, wherein the user U may also adapt the setting parameters.

Referring now to FIG. 8, by means of the switch module 24 a user U can choose between the different configurations. Hence, the user U may switch the system between the closed-loop configuration and the open-loop configuration.

Referring now to FIG. 9, generally, an index depends on the effect-site concentration Ce of a particular anesthetic agent. For example, if propofol is used, as hypnotic agent, the corresponding index A representing the level of consciousness, qCON, may generally have a value in between 0 to 100, wherein 100 indicates maximum consciousness and low values of the index indicate a reduced level of consciousness (see FIG. 9 on the left).

Similarly, if remifentanil is used as an analgesic agent, the corresponding index B, in this case, the qNOX or the qNOXenhanced, is scaled to the range between 0 to 100 and indicates the response to noxious stimuli (see FIG. 9 on the right). From FIG. 9 it can be seen that both indices are functions of their corresponding effect-site concentrations. The functions shown, index=θ(Ce), are obtained fitting the Hill equation from an extensive dataset of thousands of patients undergoing GA under propofol and remifentanil.

Referring now to FIG. 10, different anesthetic agents may exhibit a different effect-site concentration, and indices relating to the different anesthetic agents and indicating an effect associated with the particular anesthetic agent exhibit a different dependency on a respective effect-site concentration. Similarly, combined dose-response, FIG. 11, and its equivalent index-response bivariate functions, and their parameterization are obtained by fitting the Minto's interaction model or other sigmoidal-wise bivariate function on extensive datasets. The dose-response interaction parameters for different drug combinations are reported in the literature for different datasets (Boullion 2004, Kern 2004). The index-response function I is obtained by substituting the doses by the indexes in the Hill interaction parametric model using the independent Hill dose-response equations.

Generally, the specific effect of a particular anesthetic agent as indicated by the index relating to the particular anesthetic agent may be modelled by a Hill equation:

$$Effect = θ ( C e ) = {E}_{0} - \frac{{E}_{max} {C}_{e}^{γ}}{{C}_{50}^{γ} + {C}_{e}^{γ}}$$

The Hill equation herein is adapted for the different anesthetic agents, such that each anesthetic agent causes a different specific effect (index A, index B index C in FIG. 10) based on a particular effect-site concentration Ce of that particular anesthetic agent.

In addition, if multiple anesthetic agents are administered to the patient P in combination, the different anesthetic agents generally interact. This can be modelled using the anaestetics interaction model (H), such as the parametric Minto's interaction model as follows:

$$Effect = H ( C {e}_{A} , {Ce}_{B} ) = \frac{{( \frac{{C}_{A} + {C}_{B}}{{C}_{50} ( T )} )}^{γ}}{1 + {( \frac{{C}_{A} + {C}_{δ}}{{C}_{50} ( T )} )}^{γ}}$$

$$where$$

$$T = ( {C}_{A} ) / ( {C}_{A} + {C}_{B} )$$

$${C}_{50} ( T ) = 1 - β T + β {T}^{2}$$

where C_{A}, C_{B} are the normalized effect-site concentrations of the anesthetic agents A and B to their respective potency concentration, γ (>0) is the steepness of the relation between the drug combination and the effect measured as probability, and β describes the interaction strength. The anaestetics interaction model describes the interaction strenght for all the possible concentrations. There are several ways to quantify the interaction as a combined effect, the most commonly used is as the probability of response to external stimulation, ranging from 0 to 1, where 1 means a certain lack of response to the stimulus. The higher the drug concentrations the higher probability that patients do not respond to external stimulation.

This is illustrated in FIG. 11, showing the 2D surface interaction model (H) as a function of the effect-site concentrations of drug A and drug B at some probability response values. This is an example of a synergistic drug interaction model, where the contour levels of H have a hyperbola shape, wherein the same effect is achieved with lower drug concentrations when both drugs are combined.

When interpretating FIG. 11 it has to be kept in mind, that a high index corresponds to a low effect and vice versa. Hence, the point where the x-axis meets the y-axis corresponds to index A=100 and index B=100, meaning that a patient is awake.

The effect of the combination of the drugs, or the overall anesthetic effect is given at the pairs of solid and dotted lines, which are namely labeled with 0.90, 0.95 and 0.99 (meaning 0.90, 0.95 and 0.99). The overall anesthetic effect ranges between 0.0 and 1, and it gives a value for the probability that a patient does not response to the stimulus. In other words, a value close to 0.0 means that a patient is awake, wherein a value close to 1 means that a patient is in deep anesthesia. Thus, the pair of solid and dotted lines labeled with 0.90 correspond to a lower overall anesthetic effect than the pair labeled with 0.95 and 0.99.

For example, a target (overall anesthetic) effect of 0.95 is to be reached. This corresponds to a level of anesthesia where a patient is in a safe range. One would avoid 0.99 as a target effect because of the risk of overdosage. The target effect can be reached with different combinations of Index A and Index B, following the lines. For example, a high overall effect can be reached by a combination of a low Index B with a high Index A and vice versa. Considering such 2D interaction model gives a practitioner enhanced possibilities to define a proper index target for the different drugs in order to reach the desired overall effect. This might for example be helpful in case a patient is more sensitive to one drug than to the other.

If parameter β>0, there is a synergistic interaction between drug A and drug B. Changes in the concentration of drug A, potentiating effect A, will have some effects in index B, potentiating also its corresponding effect, and vice versa. Particularly, drugs like the hypnotic propofol and the analgesic remifentanil have a synergistic effect. If β<0, both drugs have an antagonist behaviour. Changes in the concentration of drug A, potentiating effect A, will have some effects in index B by reducing effect B, and vice versa.

The anaesthetics interaction models, which are reported in the literature for different drug combinations as parametric surfaces such as the Minto's interaction model, serve to enhance the quality of the control actions that performing the control actions on each drug separately, facilitating faster times to reach the indexes targets as well as stability.

The Hill equations for the different anesthetic agents as well as the interaction models are adjusted in advance from extensive datasets, such that the Hill equations and the interaction models are predefined in the system.

FIG. 12A shows the Hill index-concentration functions for two drugs, A and B modeled independently. In each index-response it is shown its desired index target, index (target), and its range or tolerance, beside the current index value describing the current patient effect state. The error control signals shown in FIG. 12A, errorθ_{A} or errorθ_{B} to control each corresponding pump, defined below, will be proportional to the distance between the current index value and the target.

While FIG. 12A represents the operation of an independent control system per drug, FIG. 12B represents the control system based on the interaction effect of the drugs modeled with the Minto's interaction function. Similarly, the practitioner defines the desired index targets to be compared with the current index values. As the interaction between two drugs is modelled in the literature in concentration coordinates, H(Ce_{A}, Ce_{B}), the error signals are defined under different coordinate's transformations between concentrations and indexes, as depicted in FIG. 12B.

A target is defined for the different effects caused by the different anesthetic agents, the target being expressed by for example a desired range of the index associated with each anesthetic agent. Hence, in the general example of FIG. 12A, a desired range for index A is defined, and in addition a desired range for index B is defined, the control operation of the control device 2 being implemented to adjust operation of the infusion devices 31-33 to cause the indices to converge to their predefined target regions. For instance, the hypnotic effect under propofol and its associated effect-index quantified via the qCON index can be set to a moderate hypnosis, a range between 60-80, while the analgesic effect induced with remifentanil and quantified with the enhanced version of the qNOX can be set e.g. in a surgical context to a depth analgesia protection, corresponding to a range between 30-40. The target region will be the intersect area in FIG. 12 with its corresponding area on the aesthetic interaction model. The target region behaves as the tolerance deviation accepted in the controller from the center point target, in this example case the target point (70, 35). Referring now to FIG. 13, the controller module 23 is adapted to compute the setting parameters for adapting the operation of the infusion devices 31-33 based on the indices as computed by the extraction module 21.

In particular, target values in the shape of target regions for the different indices are fed to the controller module 23, in addition to actual, momentary values for the indices, for example index A and index B (e.g. qCON and qNOX) and both effect site concentrations given by the PKPD models.

The controller module 23 uses a four-component error vector signal as its input. Two of the input vector components are obtained from the inverse of the Hill equation for each index and drug independently, where the error signal accounts for the difference between the desired and measured effect per drug independently. These two independent component error signals, named as errorθ_{A} and error errorθ_{B}, are:

$$\begin{matrix}{error θ}_{A} = d ( {θ}_{A}^{- 1} ) = ( index ( A ) - target ( A ) ) \frac{d {θ}^{- 1} ( index A )}{d index A} \\ {error θ}_{B} = d ( {θ}_{B}^{- 1} ) = ( index ( B ) - target ( B ) ) \frac{d {θ}^{- 1} ( index B )}{d index B}\end{matrix}$$

The other error components account for the difference between the desired and measured effect over the interaction map, and therefore, considering in the error signals the interaction effect between the two drugs (synergistic or antagonist).

These two error components (or interaction components) are functions of the two components of the gradient vector at the current (index A, index B) point on the interaction map.

The gradient vector T is defined simpler at the (index A, index B) point as

$$T = ( {T}_{1} , {T}_{2} ) = ( \frac{\partial H}{\partial index A} , \frac{\partial H}{\partial index B} )$$

Consequently, the gradient vector is transformed into the same output space as the first two error components, errorθ_{A} and error errorθ_{B}, by means of the Hill equation, resulting in the third and fourth error components, namely errorH_{A} and error errorH_{B}

$$\begin{matrix}error {H}_{A} = ( index ( A ) - target ( A ) ) \frac{d {θ}^{- 1} ( {T}_{1} )}{d index A} \\ error {H}_{B} = ( index ( B ) - target ( B ) ) \frac{d {θ}^{- 1} ( {T}_{2} )}{d index A}\end{matrix}$$

The four components of the error vector are

e=[errorθ_{A}, errorθ_{B}, errorH_{A}, errorH_{B}]

After the calculation of the four-component error vector, this vector signal is fed to a multi-variate controller, either implemented with a multi-variate PID (Proportional Integral and Derivative) controller or using a LQR (Linear Quadratic Regulator). The controller provides the new target effect actions to the pumps. The controller is accompanied by a set of limiting safety constraints, which shall limit abrupt accidental overdosing dosing rates. At least some of the limiting safety constraints values can be redefined by the practitioner.

The parameters of the controller, under normal operation with two drugs, rely mainly on the third and fourth components, representing the interaction components of the error vector, which have a higher weight in the controller constant parameter definitions. The interaction components provide better control of the two drugs because they take into account bind effects between the drugs.

When one of the pumps is not operative, the controller operation changes automatically to a uni-variate control operation mode where only the independent error component of the working pump is used.

In the controller module 23, using an inverse Hill equation for each index, the effect-site concentration of the anesthetic agent associated with the respective index is computed. In addition, using an inverse Minto equation an interaction of the different anesthetic agents is taken into account for computing the effect-site concentration, e.g. by correcting the effect-site concentrations as computed by the inverse Hill equations. Using the effect-site concentrations determined in this and way by employing the pharmacokinetic/pharmacodynamic model for each anesthetic agent, then, setting parameters may be determined for controlling operation of the infusion devices 31-33, based on error signal derived from a deviation of the actual index values from the target index values and by using, for example, a PID controller, in combination with a Linear Quadratic Regulator (LQR). In addition, a limit control may be employed to bound limits to reasonable values, in particular, to avoid excessive changes and overshooting.

Based on setting parameters determined by the controller module 23, the infusion devices 31-33 are controlled, wherein the setting parameters are forwarded to the infusion devices 31-33 in a closed-loop configuration or, upon user confirmation, in an open-loop configuration. By means of bio-signals derived from the patient P, indices are repeatedly computed anew, and the setting parameters are continuously adjusted in order to control the operation of the infusion devices 31-33.

By controlling the infusion devices 31-33 based on indices and hence on parameters indicating an effect of different anesthetic agents, an effect-based control of the infusion devices 31-33 to achieve a desired, combined effect of different anesthetic agents is implemented.

As said, for the processing non-linear models in the shape of fuzzy logic models or quadratic equation models may be employed for computing different indices, in particular the index of consciousness, qCON, and the index of nociception, qNOX. However, also other non-linear models may be used.

In the following, by way of example details about ANFIS models and quadratic equation models are provided.

### ANFIS Model

A fuzzy logic model may for example act as the ANFIS model. In that case, the system uses ANFIS models to combine the parameters, for the definition of the qCON and qNOX indices. The parameters extracted from the EEG signals are used as input to an Adaptive Neuro Fuzzy Inference System (ANFIS).

ANFIS is a hybrid between a fuzzy logic system and a neural network. ANFIS does not assume any mathematical function governing the relationship between input and output. ANFIS applies a data-driven approach where training data decides the behaviour of the system.

The five layers of ANFIS, shown in FIGS. 14A and 14B, have the following functions:
Each unit in Layer 1 stores three parameters to define a bell-shaped membership function. Each unit is connected to exactly one input unit and computes the membership degree of the input value obtained.
Each rule is represented by one unit in Layer 2. Each unit is connected to those units in the previous layer, which are from the antecedent of the rule. The inputs into a unit are degrees of membership, which are multiplied to determine the degree of fulfilment for the rule represented.
In Layer 3, for each rule, there is a unit that computes its relative degree of fulfilment by means of a normalisation equation. Each unit is connected to all the rule units in Layer 2.
The units of Layer 4 are connected to all input units and to exactly one unit in Layer 3. Each unit computes the output of a rule.
An output unit in Layer 5 computes the final output by summing all the outputs from Layer 4.

Standard learning procedures from neural network theory are applied in ANFIS. Back-propagation is used to learn the antecedent parameters, i.e. the membership functions, and least squares estimation is used to determine the coefficients of the linear combinations in the rules' consequents. A step in the learning procedure has two passes. In the first pass, the forward pass, the input patterns are propagated, and the optimal consequent parameters are estimated by an iterative least mean squares procedure, while the antecedent parameters are fixed for the current cycle through the training set. In the second pass (the backward pass) the patterns are propagated again, and in this pass back-propagation is used to modify the antecedent parameters, while the consequent parameters remain fixed. This procedure is then iterated through the desired number of epochs. If the antecedent parameters initially are chosen appropriately, based on expert knowledge, one epoch is often sufficient as the LMS algorithm determines the optimal consequent parameters in one pass and if the antecedents do not change significantly by use of the gradient descent method, neither will the LMS calculation of the consequents lead to another result. For example in a 2-input, 2-rule system, rule 1 is defined by

if x is A and y is B then f₁=p₁x+q₁y+r₁

where p, q and r are linear, termed consequent parameters or only consequents. Most common is f of first order as higher order Sugeno fuzzy models introduce great complexity with little obvious merit.

The inputs to the ANFIS system are fuzzified into a number of predetermined classes. The number of classes should be larger or equal two. The number of classes can be determined by different methods. In traditional fuzzy logic the classes are defined by an expert. The method can only be applied if it is evident to the expert where the landmarks between two classes can be placed. ANFIS optimizes the position of the landmarks, however the gradient descent method will reach its minimum faster if the initial value of the parameters defining the classes is close to the optimal values. By default, ANFIS initial landmarks are chosen by dividing the interval from minimum to maximum of all data into n equidistant intervals, where n is the number of classes. The number of classes could also be chosen by plotting the data in a histogram and visually deciding for an adequate number of classes, by ranking as done by FIR, through various clustering methods or Markov models. The ANFIS default was chosen for this invention and it showed that more than three classes resulted in instabilities during the validation phase, hence either two or three classes were used.

Both the number of classes and number of inputs add to the complexity of the model, i.e., the number of parameters. For example, in a system with four inputs each input may be fuzzified into three classes consisting of 36 antecedent (non-linear) and 405 consequent (linear) parameters, calculated by the following two formulas:

$$antecedents = number of classes \times number of inputs \times 3$$

$$consequents = number of classes number of inputs \times ( number of inputs + 1 )$$

The number of input-output pairs should in general be much larger (at least a factor 10) than the number of parameters in order to obtain a meaningful solution of the parameters.

A useful tool for ensuring stability is the experience obtained by working with a certain neuro-fuzzy system such as ANFIS in the context of a particular data set, and testing with extreme data for example obtained by simulation

ANFIS uses a Root Mean Square Error (RMSE) to validate the training result and from a set of validation data the RMSE validation error can be calculated after each training epoch. One epoch is defined as one update of both the antecedent and the consequent parameters. An increased number of epochs will in general decrease the training error.

### Quadratic Model

Alternatively, quadratic equation models may be used for the models M1, M2. In that case, the system uses quadratic models to combine the parameters for the definition of the qCON and qNOX indices. Parameters extracted from the EEG signals are used as inputs to a quadratic model.

The output indexes are derived from quadratic generalized models that use as inputs data extracted from the EEG. Such a model contains an independent coefficient called Intercept, one linear term per input, a square term per input and interaction terms between each pair of entries. The model can be expressed as:

$$Output = Intercept + {\sum }_{i = 1}^{n} {a}_{i} * {Input}_{i} + {\sum }_{i = 1}^{n} {b}_{i} * {Input}_{i}^{2} + {\sum }_{j = 1}^{n} {\sum }_{i = j + 1}^{n} {c}_{j , i} * {Input}_{i} * {Input}_{j}$$

Where:
Intercept: intersection or constant term.
Input: input model.
Output: model output.
n: number of model inputs
a: linear terms.
b: square terms
c: interaction terms between inputs.

### LIST OF REFERENCE NUMERALS

1 Rack
2 Control device
21 Extraction module
22 Modelling module
23 Controller module
230 Index computation module
231 Setting module
24 Switch module
31, 32, 33 Infusion device
310, 320, 330 Line
4 Ventilation device
40 Mouth piece
400 Line
41 Junction
5 Bio-signal monitor
51 EEG monitor
52 EMG monitor
53 ECG monitor
54 HEMO monitor
55 Impedance monitor
56 Stimulation device
57 Indexes measurements and targets comparator
6 Display device
7 Monitor device
A1-A5 Compartments
C Correction module
D Drug dosage
M1, M2 Model
p Model
P Patient
qCON Index of consciousness
qNOX Index of nociception
qNOXcor Corrected qNOX index
U Practioner

1. A system for administering anesthetic agents to a patient (P), the system comprising:
a bio-signal monitor for measuring at least one biological signal on the patient (P);
an arrangement of infusion devices for infusing at least a first anesthetic agent and a second anesthetic agent to the patient (P); and
a control device configured to compute, based on the at least one biological signal, a first index relating to a first effect caused by the first anesthetic agent and a second index relating to a second effect caused by the second anesthetic agent;
wherein the control device is configured to compute, based on the first index and the second index, a first setting parameter for adjusting an infusion of the first anesthetic agent and a second setting parameter for adjusting an infusion of the second anesthetic agent.

2. The system according to claim 1, wherein the first anesthetic agent is a hypnotic agent and the second anesthetic agent is an analgesic agent, wherein the first index relates to a hypnotic effect and the second index relates to an analgesic effect.

3. The system according to claim 1, wherein the first index is an index of consciousness (qCON) and the second index is an index of nociception (qNOX), or its enhanced version.

4. The system according to one of claims 1, wherein the control device is configured to provide said first setting parameter to a first of the infusion devices for controlling operation of the first of the infusion devices for infusing the first anesthetic agent and to provide said second setting parameter to a second of the infusion devices for controlling operation of the second of the infusion devices for infusing the second anesthetic agent.

5. The system according to claim 1, wherein the control device comprises a switch module which is actuatable to switch between a closed-loop configuration, in which the control device is configured to automatically output said first setting parameter to the first of the infusion devices and said second setting parameter to the second of the infusion devices, and an advisory configuration, in which the control device is configured to output said first setting parameter and said second setting parameter to a user (U) prior to transferring said first setting parameter to the first of the infusion devices and said second setting parameter to the second of the infusion devices.

6. The system according to claim 1, wherein the control device is configured to compute said first setting parameter and said second setting parameter based on a first pharmacokinetic/pharmacodynamic model relating to a pharmacokinetic/pharmacodynamic behaviour of the first anesthetic agent and a second pharmacokinetic/pharmacodynamic model relating to a pharmacokinetic/pharmacodynamic behaviour of the second anesthetic agent.

7. The system according to claim 1, wherein the control device is configured to compute said first setting parameter and said second setting parameter based on a first Hill model modelling a first effect at an effect site based on an effect-site concentration of the first anesthetic agent and a second Hill model modelling a second effect at the effect site based on an effect-site concentration of the second anesthetic agent.

8. The system according to claim 7, wherein the control device is configured to compute a value of the effect-site concentration of the first anesthetic agent using an inverse Hill equation with the first index as input, the first index being indicative of the first effect, and a value of the effect-site concentration of the second anesthetic agent using an inverse Hill equation with the second index as input, the second index being indicative of the second effect.

9. The system according to claim 1, wherein the control device is configured to compute said first setting parameter and said second setting parameter based on an interaction model modelling a combined effect of an effect-site concentration of the first anesthetic agent and an effect-site concentration of the second anesthetic agent.

10. The system according to claim 1, wherein the biosignal monitor comprises at least one of an EEG module for measuring an electroencephalogram signal on the patient (P), an EMG monitor for measuring an electromyogram signal on the patient (P), an ECG monitor for measuring an electrocardiogram signal, a hemodynamics monitor for measuring at least one signal relating to hemodynamics of the patient (P), an impedance monitor for measuring an impedance on the patient (P), a piezoelectric sensor, and a skin response sensor.

11. The system according to claim 1, wherein the biosignal monitor comprises a stimulation device for applying at least one stimulus to the patient (P).

12. The system according to claim 11, wherein the stimulation device is configured to apply different stimuli to different locations of the patient (P) and configured to apply an electrical stimulus according to a predefined stimulation pattern.

13. The system according to claim 1, wherein the control device is configured to compute, based on at least one biological signal, a third index relating to a third effect caused by a third anesthetic agent infused by said arrangement of infusion devices, and to compute, based on the third index, a third setting parameter for adjusting an infusion of the third anesthetic agent.

14. The system according to claim 1, wherein the control device contains a multivariate PID or LQR controller on the setting parameters prior the pumps.

15. A method for administering anesthetic agents to a patient (P), the method comprising:
measuring at least one biological signal on the patient (P) using a bio-signal monitor;
infusing at least a first anesthetic agent and a second anesthetic agent to the patient (P) using an arrangement of infusion devices; and
computing, using a control device and based on the at least one biological signal, a first index relating to a first effect caused by the first anesthetic agent and a second index relating to a second effect caused by the second anesthetic agent;
wherein said computing further comprising, using the control device and based on the first index and the second index, a first setting parameter for adjusting an infusion of the first anesthetic agent and a second setting parameter for adjusting an infusion of the second anesthetic agent.
